(19) United States
(12) Patent Application Publication
(10) Pub. No.: US 2025/0339073 A1
(43) Pub. Date: Nov. 6, 2025

(54) Automated Urinary Output-Measuring Systems and Methods

(71) Applicant: C. R. Bard, Inc., Franklin Lakes, NJ (US)

(72) Inventors: Jason Jishen Cheng, Avondale Estates, GA (US); Audrey Earnshaw, Erie, CO (US); Trevor Dimicco, Providence, RI (US); Juan C. Tapia, Larenceville, GA (US)

(21) Appl. No.: 19/268,144

(22) Filed: Jul. 14, 2025

Related U.S. Application Data
(63) Continuation of application No. 17/556,931, filed on Dec. 20, 2021, now Pat. No. 12,364,423.
(60) Provisional application No. 63/128,558, filed on Dec. 21, 2020.

(51) Int. Cl. A61B 5/20; A61B 5/00; A61B 10/00; G01F 22/00

(52) U.S. Cl. CPC A61B 5/208; A61B 5/0004; A61B 5/6807; A61B 5/7246; A61B 10/007; G01F 22/00; A61B 2562/0219

(57) ABSTRACT

An automated urinary output (“UO”)-measuring system includes a fluid container including a console coupled to one or more ultrasonic sensors and one or more accelerometers. The console includes a processor configured to receive accelerometer values from the one or more accelerometers, to determine an acceleration state of the fluid container, to activate the one or more ultrasonic sensors when the acceleration state of the fluid container is below a threshold, and to measure a volume of fluid in the fluid container when the acceleration state of the fluid container is zero. The system can further include a removable valve coupled to the fluid container and an attachment device including a pocket configured to accommodate the fluid container.

### PRIORITY

This application is a continuation of U.S. patent application Ser. No. 17/556,931, filed Dec. 20, 2021, now U.S. Pat. No. 12,364,423, which claims the benefit of priority to U.S. Provisional Application No. 63/128,558, filed Dec. 21, 2020, each of which is incorporated by reference in its entirety into this application.

### BACKGROUND

Low urinary output in those with congestive heart failure (“CHF”) can be a symptom of low cardiac output. It can be difficult to non-invasively measure urinary output in those with CHF as they are often ambulatory, and most automated urinary output measuring devices are invasive and engineered for the intensive care unit and non-ambulatory patients. It would be beneficial to CHF patients and clinicians to be able to measure urinary output accurately and automatically in ambulatory CHF patients.

Disclosed herein are automated urinary output (“UO”)-measuring systems and methods that address the foregoing.

### SUMMARY

Disclosed herein is an automated UO-measuring system including a container configured to collect fluid, the container having a console, one or more ultrasonic sensors coupled to the console, one or more accelerometers coupled to the console, and a valve configured to pass fluid therethrough. The system also includes a fluid line coupled to the valve and a container holder. The container holder has a sleeve configured to be secured to a user and a pocket configured to securely hold the container.

In some embodiments, the console, the one-or-more ultrasonic sensors, the one-or-more accelerometers, and the valve are organized into a panel.

In some embodiments, the panel divides the container into a proximal section or a distal section.

In some embodiments, the panel is located at a proximal end or a distal end of the container.

In some embodiments, the panel includes a pump configured to create a low-pressure environment inside the container.

In some embodiments, the console includes one of more processors, a non-transitory storage medium, an energy source and one or more logic modules.

In some embodiments, the one-or-more logic modules are configured to receive accelerometer values from the one-or-more accelerometers, determine an acceleration state of the container, activate the one-or-more ultrasonic sensors, receive ultrasonic sensor values from the one-or-more ultrasonic sensors, correlate the ultrasonic sensor values with a volume-of-voided-urine value within the container and a time-of-day value for a correlation, determine a volume of urine using the ultrasonic sensor values, activate a pump to create and maintain a low-pressure environment inside the container, transmit the correlation to a computing device, or a combination thereof.

In some embodiments, the one-or-more logic modules are configured to activate the one-or-more ultrasonic sensors occurs when the acceleration state of the container is below a threshold.

In some embodiments, the container holder is configured to be detachably secured to the user.

In some embodiments, the sleeve includes two or more arms configured to wrap around an appendage of the user.

In some embodiments, the two-or-more arms are organized into a first pair of fastening arms and a first pair of securing arms.

In some embodiments, the container holder is secured to the appendage of the user by hook-and-loop fasteners or magnets.

In some embodiments, the sleeve includes a compression sock configured to be slidably secured to an appendage of the user.

In some embodiments, the container includes a rigid container.

Also disclosed herein is a method of automatically measuring urine output including capturing a volume of voided urine from a user, in a container using a fluid line, the container being coupled to the user, distal a bladder of the user, the container having a valve configured to pass fluid therethrough and a console coupled to one or more ultrasonic sensors and one or more accelerometers. The method also includes detecting an acceleration state of the container, measuring the volume of voided urine over time in the container, correlating the measured volume of voided urine with a volume value and a time-of-day value, and transmitting the volume value and the time-of-day value to a computing device.

In some embodiments, capturing the volume of voided urine from the user includes maintaining a low-pressure environment in the container with a pump of the container.

In some embodiments, detecting the acceleration state of the container includes using the one-or-more accelerometers to detect the acceleration state of the container.

In some embodiments, measuring the volume of voided urine over time in the container includes measuring the volume when the acceleration state of the container is zero.

In some embodiments, measuring the volume of voided urine over time in the container includes using the one-or-more ultrasonic sensors to measure the volume of voided urine over time.

In some embodiments, measuring the volume of voided urine over time in the container includes both measuring and recording at evenly spaced time intervals.

In some embodiments, the time intervals are user-defined.

In some embodiments, transmitting the volume value and the time-of-day value to the computing device includes wirelessly transmitting the volume value and the time-of-day value to the computing device.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the automated UO-measuring systems and methods will be rendered by reference to specific embodiments thereof that are illustrated in the drawings. It is appreciated that these drawings depict only some embodiments of the foregoing and are, therefore, not to be considered limiting to the scope of the concepts provided herein. Example embodiments will be described and explained with additional specificity and detail through the use of the accompanying drawings in which:

FIG. 1A illustrates a perspective view of an automated UO-measuring system in accordance with some embodiments.

FIG. 1B illustrates a perspective view of the automated UO-measuring system in accordance with some embodiments.

FIG. 1C illustrates a side view of a container of the automated UO-measuring system in accordance with some embodiments.

FIG. 2 illustrates a block diagram of some components of the automated UO-measuring system in accordance with some embodiments.

FIG. 3A illustrates a method of measuring urinary output in accordance with some embodiments

FIG. 3B illustrates another method of measuring urinary output in accordance with some embodiments.

FIG. 4A illustrates a container holder of the automated UO-measuring system in a ready-to-use state in accordance with some embodiments.

FIG. 4B illustrates the container holder of FIG. 4A in use in accordance with some embodiments.

FIG. 5A illustrates a side view of another container holder of the automated UO-measuring system in use in accordance with some embodiments.

FIG. 5B illustrates a front view of the container holder of FIG. 5A in use in accordance with some embodiments.

FIG. 6 illustrates a flow chart of a method of measuring urinary output using the automated UO-measuring system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a container disclosed herein includes a portion of the container intended to be near a clinician when the container is used on a user. Likewise, a “proximal length” of, for example, the container includes a length of the container intended to be near the clinician when the container is used on the user. A “proximal end” of, for example, the container includes an end of the container intended to be near the clinician when the container is used on the user. The proximal portion, the proximal-end portion, or the proximal length of the container can include the proximal end of the container; however, the proximal portion, the proximal-end portion, or the proximal length of the container need not include the proximal end of the container. That is, unless context suggests otherwise, the proximal portion, the proximal-end portion, or the proximal length of the container is not a terminal portion or terminal length of the container.

With respect to “distal,” a “distal portion” or a “distal-end portion” of, for example, a container disclosed herein includes a portion of the container intended to be near or in a user when the container is used on the user. Likewise, a “distal length” of, for example, the container includes a length of the container intended to be near or in the user when the container is used on the user. A “distal end” of, for example, the container includes an end of the container intended to be near or in the user when the container is used on the user. The distal portion, the distal-end portion, or the distal length of the container can include the distal end of the container; however, the distal portion, the distal-end portion, or the distal length of the container need not include the distal end of the container. That is, unless context suggests otherwise, the distal portion, the distal-end portion, or the distal length of the container is not a terminal portion or terminal length of the container.

Alternatively, logic can be software, such as executable code in the form of an executable application, an Application Programming Interface (API), a subroutine, a function, a procedure, an applet, a servlet, a routine, source code, object code, a shared library/dynamic load library, or one or more instructions. The software can be stored in any type of a suitable non-transitory storage medium, or transitory storage medium (e.g., electrical, optical, acoustical or other form of propagated signals such as carrier waves, infrared signals, or digital signals). Examples of non-transitory storage medium can include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code can be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN,” etc.), or a combination of networks. Examples of a computing device can include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message can be in the form of one or more packets, frames, HTTP-based transmissions, or any other series of bits having the prescribed format.

Unless defined otherwise, all technical and scientific terms used herein have the same meaning as commonly understood by those of ordinary skill in the art.

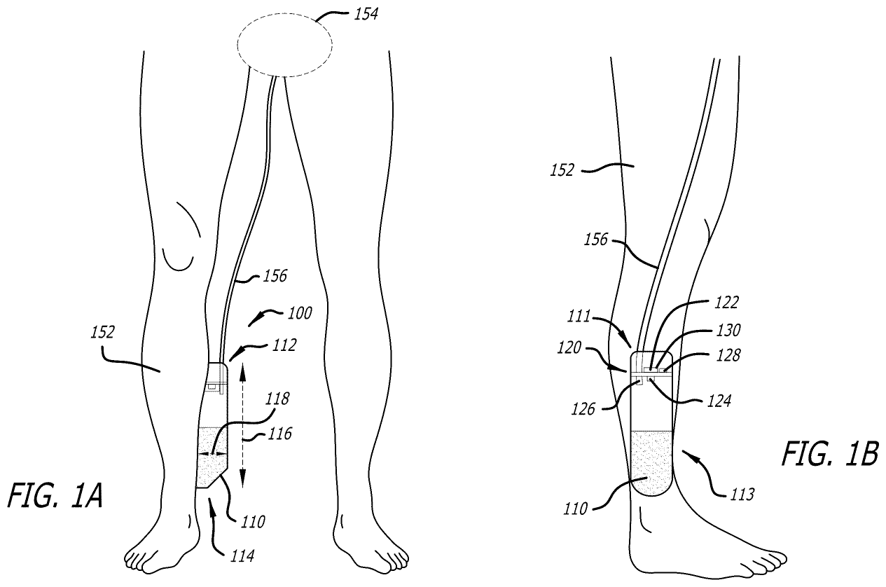

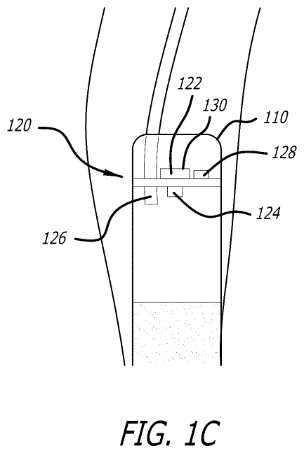

FIG. 1A illustrates a perspective view of an automated UO-measuring system 100 in accordance with some embodiments. In some embodiments, the automated UO-measuring system 100 includes a container 110 configured to receive and contain a volume of voided urine generated by a user or patient. In some embodiments, the container 110 can be a rigid container, which includes a container length 116, a container depth 118 and is configured to contain a volume of fluid therein. As used herein, a rigid container means a container that is stiff and unyielding as opposed to pliant or flexible. In some embodiments, the container 110 is clear, for visual determination of the fluid volume therein. The container 110 can be configured to have various shapes including a triangular prism, a rectangular prism, a pentagonal prism, an irregular prism, a cylinder, a polyhedron or the like. In some embodiments, the container 110 has a fixed three-dimensional structure. In an embodiment, the container 110 includes a cavity configured to fit the container 110 flush against a user's appendage 152. In some embodiments, the container 110 can be constructed of a hardened polymer such polycarbonate, polyethylene, polypropylene, polystyrene or the like.

In some embodiments, the container 110 includes a valve 126 on a proximal end 112 of the container 110. In some embodiments, the valve 126 can include a directional valve, a check valve, umbrella valve, flapper valve or the like. In some embodiments, the valve 126 can be configured to be removed, to dispose of the volume of voided urine in the container 110. In some embodiments, a fluid line 156 from the user configured to transport voided urine therein, can be distally coupled to the valve 126 of the container 110. In some embodiments, the fluid line 156 can include a hollow tubing constructing of a clear plastic polymer such as polycarbonate, polyethylene terephthalate, polystyrene, urethane, nylon or the like. In some embodiments, the fluid line 156 can be coupled to a urine collection device, wherein the urine collection device is configured to capture a volume of voided urine from the user's bladder 154 and the fluid line 156 is configured to channel the urine to the container 110 through the valve 126. The container 110 is configured to be secured to a user, distal of the user's bladder 154 in order to allow fluid flow to the container 110 through passive gravity flow. For example, in some embodiments, the container 110 can be secured to a thigh, a calf or an ankle.

FIG. 1B illustrates a perspective view of the automated UO-measuring system 100 in accordance with some embodiments. In some embodiments, the container 110 includes the valve 126, one or more accelerometers 122, one or more ultrasonic sensors 124 and a console 130. In some embodiments, the valve 126, the one-or-more accelerometers 122, the one-or-more ultrasonic sensors 124 and the console 130 can be organized into a panel 120. In some embodiments, the panel 120 can be proximally located or distally located on the container 110. The one-or-more accelerometers 122 can be configured to detect when the container 110 is accelerating or not such that the fluid level within the container 110 can be determined when the container 110 is not accelerating. The one-or-more ultrasonic sensors 124 can be configured to detect the fluid level within the container 110 that will be described in more detail herein. The console 130 can be configured to receive accelerometer values from the one-or-more accelerometers 122, receive detected ultrasonic measurements from the one-or-more ultrasonic sensors 124 and transmit the measured or determined values in a message to a computing device that will be described in more detail herein. In some embodiments, the computing device can include a computing device, a smartphone, a medical device, a laptop or the like.

In some embodiments, the panel 120 can be configured to divide the container 110 into a proximal section 111 and a distal section 113. The container 110 can be configured to detachably separate at the panel 120, into the proximal section 111 and the distal section 113, and can be configured to be rejoined into one piece through a press fit, a snap fit, an interference fit or the like. In some embodiments, the container 110 can be configured to detachably separate to dispose of the volume of voided urine. In some embodiments, the panel 120 can be secured within the proximal section 111. In some embodiments, the fluid line 156 can be detached from the valve 126 to dispose of the volume of voided urine through the valve 126.

FIG. 1C illustrates a side view of the container 110 including the panel 120 of the automated UO-measuring system 100 in accordance with some embodiments. In some embodiments, the panel 120 includes a pump 128 configured to evacuate air from the container 110 to create a low-pressure environment inside the container 110 to assist urine drainage into the container 110. In some embodiments, the pump 128 is coupled to the console 130 and controlled by the console 130. In some embodiments, the pump 128 can be configured to be activated after the volume of voided urine within the container 110 has be disposed. In some embodiments, the pump 128 includes a pressure sensor configured to detect the pressure within the container 110 in order to maintain a consistent low-pressure environment in the container 110.

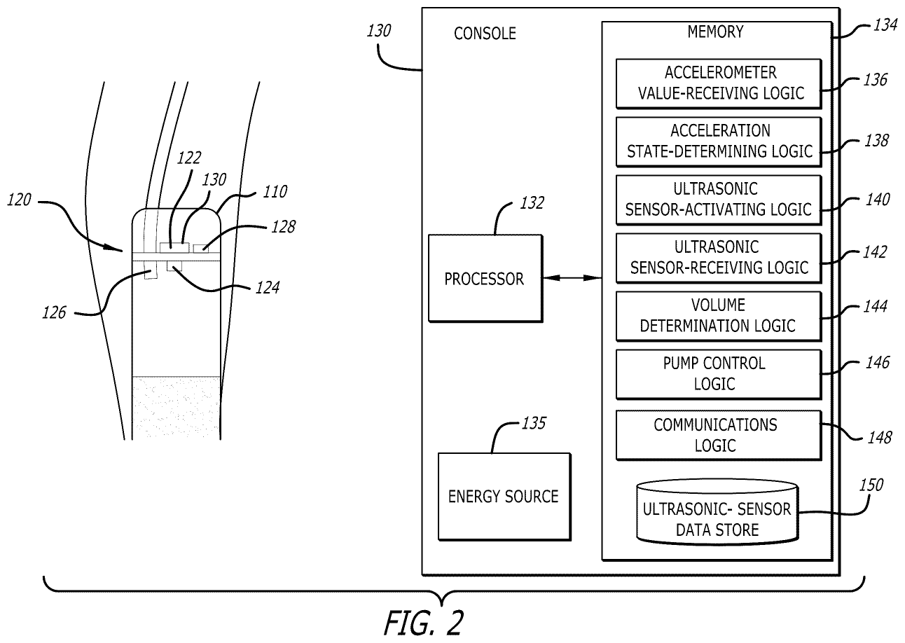

FIG. 2 illustrates a block diagram of some components of the automated UO-measuring system 100 in accordance with some embodiments. In some embodiments, the automated UO-measuring system 100 includes the console 130. In some embodiments, the console 130 can be contained within the panel 120 or coupled separately to the container 110. The console 130 includes one or more processors 132, non-transitory storage medium (“memory”) 134, an energy source 135 and one or more logic modules such as a plurality of logic modules. In some embodiments, the energy source 135 can be configured to provide energy to the one-or-more accelerometers 122, the one-or-more ultrasonic sensors 124, the pump 128, and the console 130. In some embodiments, the console 130 can be configured to detect data and transmit the detected data to a computing device for processing. In some embodiments, the one-or-more logic modules are selected from an accelerometer value-receiving logic 136, an acceleration state-determining logic 138, an ultrasonic sensor-activating logic 140, an ultrasonic sensor-receiving logic 142, a volume determination logic 144, a pump control logic 146, and a communications logic 148. In some embodiments, the memory 134 can include a data store such as an ultrasonic-sensor data store 150. The accelerometer value-receiving logic 136 can be configured to receive measured accelerometer values from the one-or-more accelerometers 122. In some embodiments, the acceleration state-determining logic 138 can be configured to determine an acceleration state of the container 110 based on the measured accelerometer values. In some embodiments, the acceleration state-determining logic 138 can determine the acceleration state of the container 110 by determining if the accelerometer values are above or below a near-zero threshold accelerometer value. In some embodiments, the ultrasonic sensor-activating logic 140 can be configured to activate the one-or-more ultrasonic sensors 124. In some embodiments, the ultrasonic sensor-activating logic 140 can be configured to activate the one-or-more ultrasonic sensors 124 only when the console 130 determines the container 110 has an acceleration state that is about zero, for example, by way of comparison to the near-zero threshold accelerometer value. In some embodiments, the ultrasonic sensor-receiving logic 142 can be configured to receive a measured time value of the time it takes an ultrasonic wave generated by the one-or-more ultrasonic sensors 124 to be detected after reflection inside the container 110, that will be described in more detail herein.

In some embodiments, the volume determination logic 144 can be configured to determine the volume of voided urine contained within the container 110 by correlating the measured time value of the reflected ultrasonic wave with a volume value corresponding to the volume of voided urine within the container 110. In some embodiments, the volume determination logic 144 can be further configured to associate a time-of-day value with each the volume value at the time of day the volume value was determined. In some embodiments, the volume determination logic 144 can be configured to generate an associated pairing of the {time-of-day value, volume value}. In some embodiments, the volume determination logic 144 can be configured to associate other parameters with the associated pairing in an associated trio, an associated quartet, an associated quintet, and an associated sextet or the like. For example, the volume determination logic 144 can associate a device-operating-condition value, a voided number in a user-defined timer-period value, a device-status value or the like. In some embodiments, the pump control logic 146 can be configured to activate the pump 128 to create the low-pressure environment within the container 110. In some embodiments, the pump control logic 146 can be configured to activate the pump 128 to maintain the low-pressure environment within the container 110. In an embodiment, the pump 128 includes the pressure sensor configured to detect the pressure within the container 110 and acquire pressure readings within the container 110. In this embodiment, the pressure sensor can transmit the pressure readings to the console 130 and the pump control logic 146 can be configured to activate the pump 128 to maintain a consistent low-pressure environment within the container 110. In some embodiments, a low-pressure environment within the container 110 can be configured to help draw fluid into the container 110.

The ultrasonic-sensor data store 150 can be configured to store the volume values, the measured time values from the one-or-more ultrasonic sensors 124, the time-of-day values, the device-status value, the device-operating-condition value, the voided number in the user-defined time-period value or a combination thereof. In some embodiments, the ultrasonic-sensor data store 150 can store the volume values and time-of-day values as the associated pairings of {time-of-day value, volume value}. In some embodiments, the communications logic 148 can be configured to transmit each associated pairing of {time-of-day value, volume value} to a computing device, an electronic medical record (“EMR”) system or the like. The communications logic 148 can be configured to wirelessly transmit the associated pairings of {time-of-day value, volume value} to the computing device. Wireless communication modalities can include Wi-Fi, Bluetooth, Near Field Communications (NFC), cellular Global System for Mobile Communication (“GSM”), electromagnetic (EM), radio frequency (RF), combinations thereof, or the like.

In some embodiments, the one-or-more accelerometers 122 can be configured to detect acceleration of the container 110 at regular timed intervals (e.g., every five minutes, every hour, every 30 seconds, or the like). In some embodiments, the one-or-more accelerometers 122 can be configured to detect acceleration of the container 110 at user-defined intervals. The automated UO-measuring system 100 can be configured to take a volume value every time the accelerometer value is below the near-zero threshold accelerometer value. In some embodiments, the automated UO-measuring system 100 can be configured to take a volume value when two consecutive accelerometer values are below the near-zero threshold accelerometer value. In some embodiments, the automated UO-measuring system 100 can be configured to take a volume value at either regular timed intervals or the user-defined intervals. In an embodiment, the user can define how many volume values the console 130 generates in a specific time period. For example, the user can desire 8 volume values in 8 hours and the automated UO-measuring system 100 can be configured to detect 1 volume value per hour or the automated UO-measuring system 100 can be configured to continually detect the acceleration state of the container 110 until 1 volume value is obtained within the hour time block.

In an embodiment, the one-or-more accelerometers 122 can be configured to detect accelerometer values of the container 110 at a regular timed interval of once every one hour. In this embodiment, if the one-or-more accelerometers 122 do detect accelerometer values of the container 110 greater than the near-zero threshold accelerometer value during the hour, the one-or-more accelerometers 122 can be configured to either wait until the next hour to detect accelerometer values of the container 110 or can wait a certain amount of time (e.g. 5 minutes) to commence detecting accelerometer values of the container 110.

In some embodiments, the console 130 can be configured to notify the user when the volume value of the volume of voided urine within the container 110 is approaching the maximum allowable volume within the container 110. In some embodiments, the maximum allowable volume can be the maximum allowable volume contained within the container 110 or can be the maximum volume of voided urine the container 110 can hold before the volume of voided urine expands into the proximal section 111 of the container 110. The console 130 can wirelessly send the information to the computing device to notify the user through visual or an audible signal.

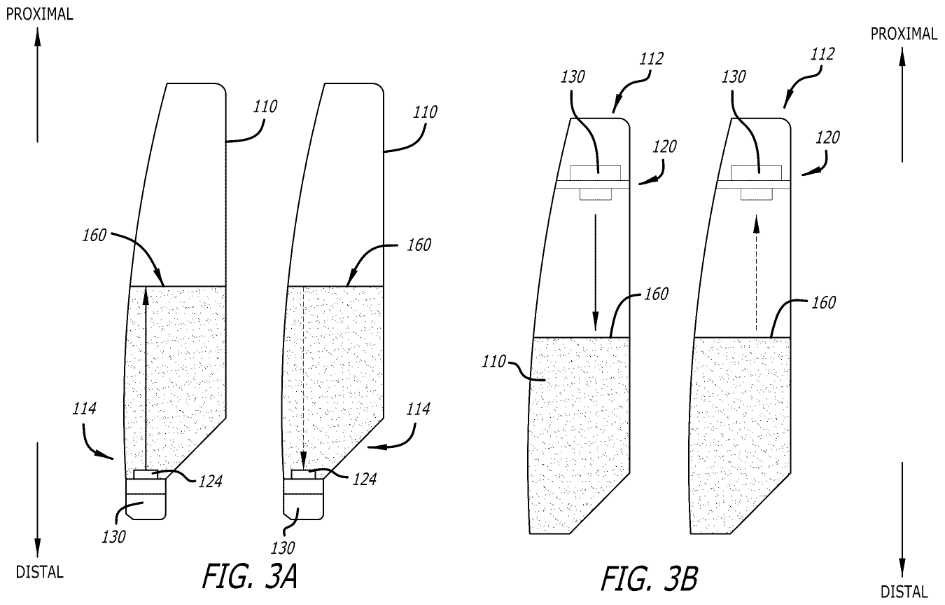

FIGS. 3A-3B illustrate methods of measuring urinary output in accordance with some embodiments. In some embodiments, as illustrated in FIG. 3A, the panel 120 including the one-or-more accelerometers 122, the one-or-more ultrasonic sensors 124 and the console 130 can be located at a distal end 114 of the container 110. The panel 120 arranges the one-or-more ultrasonic sensors 124 to be pointing proximally, towards an air/urine interface 160. The one-or-more accelerometers 122 can detect accelerometer values of the container 110 that can then be used by the console 130 to determine the acceleration state of the container 110. In some embodiments, the console 130 can be configured to activate the one-or-more ultrasonic sensors 124 only when the acceleration of the container 110 is below the near-zero threshold accelerometer value. Once the acceleration of the container 110 is below the near-zero threshold accelerometer value, the one-or-more ultrasonic sensors 124 can generate an ultrasonic wave that travels proximally through the urine until the ultrasonic wave reaches the urine/air interface 160. The ultrasonic wave is then reflected distally back through the urine, until the ultrasonic wave reaches the one-or-more ultrasonic sensors 124. The time from generation of the ultrasonic wave to the time a sensor of the one-or-more ultrasonic sensors 124 receives the reflection can be measured and transmitted to the console 130. The console 130 can be configured to send the information to the computing device to correlate the measured time to a volume value that correlates to the volume of voided urine within the container 110. In some embodiments, the console 130 can be configured to send the information to the computing device to correlate the volume value with a time-of-day value, which can be transmitted to a computing device.

As illustrated in FIG. 3B, in some embodiments, the panel 120 can be located at the proximal end 112 of the container 110. The panel 120 arranges the one-or-more ultrasonic sensors 124 to be pointing distally, towards the air/urine interface 160. Once the acceleration of the container 110 is below the near-zero threshold accelerometer value, the console 130 can be configured to activate the one-or-more ultrasonic sensors 124. The one-or-more ultrasonic sensors 124 can generate an ultrasonic wave that travels distally through the air until the ultrasonic wave reaches the air/urine interface 160. The ultrasonic wave is then reflected proximally back through the air, until the ultrasonic wave reaches the one-or-more ultrasonic sensors 124. The time from generation of the ultrasonic wave to the one-or-more ultrasonic sensors 124 receiving the reflection of the ultrasonic wave can be measured and transmitted to the console 130. The console 130 can be configured to correlate the measured time to a volume value of the volume of voided urine in the container 110. The console 130 can also be configured to correlate the volume value with a time-of-day value, which can be transmitted to a computing device.

In an embodiment, the valve 126 can be located at the proximal end 112 of the container 110 and the panel 120 including the one-or-more accelerometers 122 and the one-or-more ultrasonic sensors 124 can be located at the distal end 114 of the container 110. The one-or-more ultrasonic sensors 124 can generate an ultrasonic wave that travels through the volume of voided urine until the wave reaches the urine/air interface 160 where it is reflected back to the one-or-more ultrasonic sensors 124.

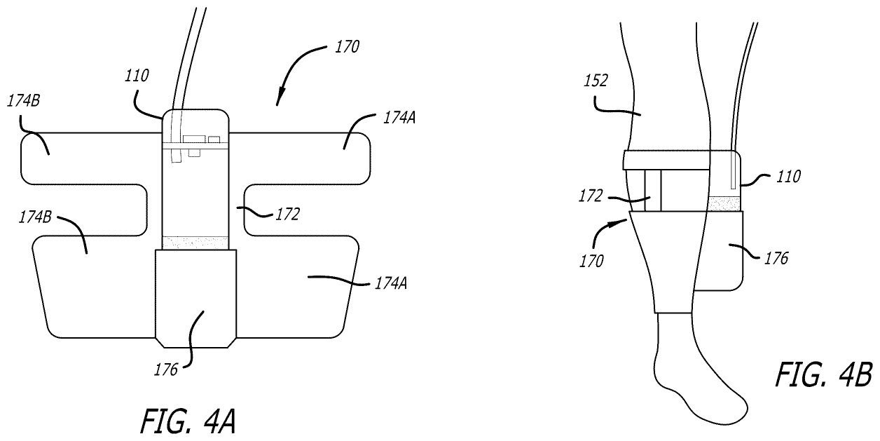

FIGS. 4A-4B illustrate a container holder (“holder”) 170 of the automated UO-measuring system 100 in accordance with some embodiments. In some embodiments, the container 110 can be secured to the user's appendage 152 by the holder 170. In some embodiments, the holder 170 can include a sleeve 172 having a pocket 176 configured to securely hold the container 110 therein. As used herein, “securely held” means held so that the container 110 is firmly positioned so as not to become easily displaced to prevent user discomfort. In some embodiments, the sleeve 172 can be constructed of one or more fabrics configured to provide a compressing force on the user's appendage 152 to prevent unwanted movement of the holder 170. In some embodiments, the pocket 176 can be constructed of one or more fabrics configured to provide a compressing force on the container 110 to prevent unwanted movement of the container 110. In some embodiments, the holder 170 can include two or more arms such as the first pair of fastening arms 174A and the first pair of securing arms 174B extending laterally from the sleeve 172. Indeed, as illustrated in FIG. 4A, the holder 170 can include a first pair of fastening arms 174A and a first pair of securing arms 174B. In some embodiments, as illustrated in FIG. 4B, the first pair of fastening arms 174A can be configured to wrap around the user's appendage 152 and be detachably secured to the first pair of securing arms 174B. In some embodiments, the first pair of fastening arms 174A can be detachably coupled to the first pair of securing arms 174B by hook-and-loop fasteners, magnets or the like. In some embodiments, the first pair of fastening arms 174A can include the hook components of the hook-and-loop fasteners and the first pair of securing arms 174B can include the loop components of the hook-and-loop fasteners. In some embodiments, the first pair of fastening arms 174A can include the loop components of the hook-and-loop fasteners and the first pair of securing arms 174B can include the hook components of the hook-and-loop fasteners.

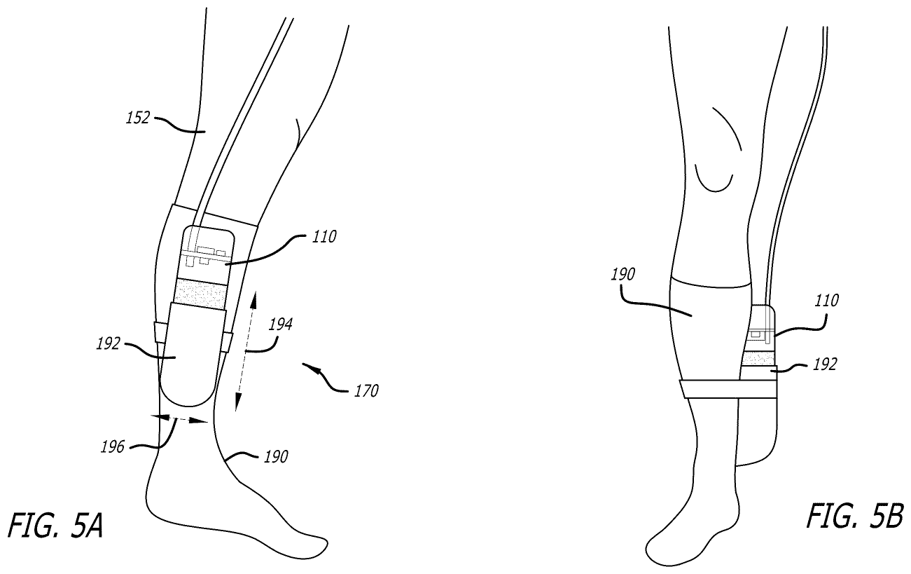

FIGS. 5A-5B illustrate different views of the holder 170 of the automated UO-measuring system 100 in accordance with some other embodiments. In some embodiments, as illustrated in FIG. 5A, the user can couple the container 110 to his or her appendage 152 by using a compression sock 190 having a pouch 192. In some embodiments, the pouch 192 can be configured to slidably receive the container 110 therein. The compression sock 190 can be configured to have the pouch 192 located medially or more offset from a midline. The compression sock 190 and the pouch 192 can be constructed from one or more fabrics that are configured to provide a compressing force on the container 110 to prevent unwanted movement of the container 110. In some embodiments, the pouch 192 has a pouch length 194 and pouch width 196. In some embodiments, the pouch length 194 can be smaller than the container 110, equal to the container 110 or greater than the container 110. Although FIGS. 4A-4B and FIGS. 5A-5B illustrate various embodiments of the holder 170 of the automated UO-measuring system 100, it can be appreciated that other methods of coupling the container 110 to the user's appendage 152 are considered.

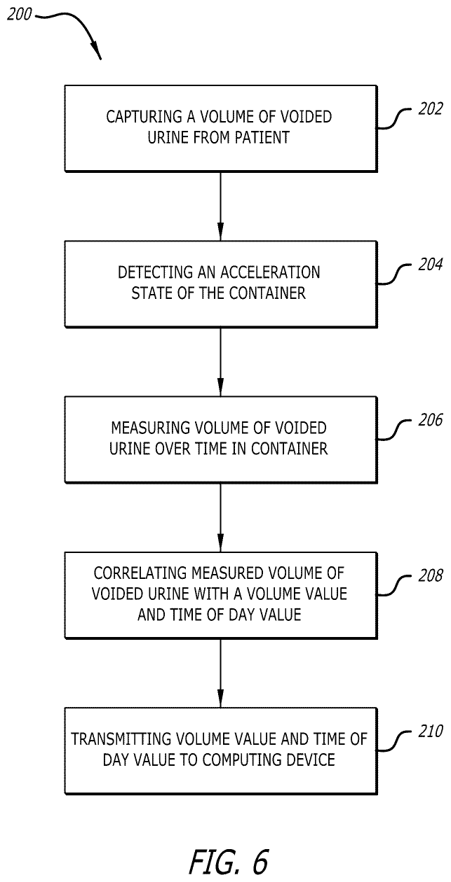

FIG. 6 illustrates a flow chart of a method 200 of automatically measuring urinary output using the automated UO-measuring system 100 in accordance with some embodiments. In some embodiments, the method 200 includes capturing voided urine from a user (block 202). In some embodiments, capturing voided urine includes using the automated UO-measuring system 100 including the container 110 configured to collect fluid therein, the container 110 having the console 130 coupled to the one-or-more accelerometers 122 and the one-or-more ultrasonic sensors 124, and having the valve 126 configured to pass fluid therethrough. In some embodiments, the container 110 is a rigid container. The automated UO-measuring system 100 further includes a fluid line 156 coupled to the valve 126. The automated UO-measuring system 100 further includes the holder 170 having the sleeve 172 configured to be coupled or secured to the user or the user's appendage 152, distal of the user's bladder 154, the sleeve 172 having the pocket 176 configured to securely hold the container 110 therein. In some embodiments, securing the container 110 with the container holder 170, distal of the user's bladder 154 allows for fluid flow into the container 110 through passive gravity flow. In some embodiments, capturing voided urine includes using a urine collection device coupled to the fluid line 156 configured to channel urine from the user's bladder 154 to the container 110. In some embodiments, capturing a volume of voided urine includes the container 110 having the pump 128 configured to maintain a low-pressure environment in the container 110. In some embodiments, maintaining a low-pressure environment within the container 110 contributes to passive fluid flow into the container 110.

In some embodiments, the method 200 includes detecting an acceleration state of the container 110 (block 204). In some embodiments, detecting the acceleration of the container 110 includes using the one-or-more accelerometers 122 to obtain accelerometer values of the container 110. In some embodiments, the accelerometer values are transmitted to the console 130 and to the computing device where the acceleration state of the container 110 can be determined by comparing the accelerometer values with the near-zero threshold accelerometer value. In some embodiments, detecting the acceleration state of the container 110 includes detecting accelerometer values at regular time intervals, user-defined intervals or continuously detecting accelerometer values.

In some embodiments, the method 200 further includes measuring the volume of voided urine over time in the container 110 (block 206). In some embodiments, measuring the volume of voided urine over time includes measuring the volume of voided urine when the acceleration state of the container 110 is below the near-zero threshold accelerometer value. In some embodiments, measuring the volume of voided urine over time in the container 110 includes generating one or more ultrasonic waves by the one-or-more ultrasonic sensors 124 that travel through the air in the container 110 until the ultrasonic waves reach the air/urine interface 160. Once reaching the air/water interface 160, the ultrasonic waves are reflected back towards the one-or-more ultrasonic sensors 124. In some embodiments, the one-or-more ultrasonic sensors 124 can generate one or more ultrasonic waves that travel through the urine in the container 110 until the ultrasonic waves reach the urine/air interface 160 where they are reflected back through the urine to the one-or-more ultrasonic sensors 124. The time from generation of the ultrasonic wave to receiving the reflected ultrasonic wave can be measured and transmitted to the console 130.

In some embodiments, the method 200 includes correlating the measured volume of voided urine with a volume value and a time-of-day value (block 208). In some embodiments, correlating the measured volume of voided urine with a volume value includes the console 130 correlating the measured time with the volume value, corresponding to the volume of voided urine within the container 110. In some embodiments, correlating the measured volume of voided urine with a volume value and a time-of-day value includes the console 130 and the computing device correlating the volume value with the time-of-day value. In some embodiments, correlating the measured volume of voided urine with a volume value and a time-of-day value includes generating an associated pairing of {time-of-day value, volume value}. In some embodiments, measuring the volume of voided urine over time includes measuring the volume of voided urine at automatically defined or user-defined time intervals.

In some embodiments, the method 200 includes transmitting the volume value and time-of-day value to a computing device (block 210). In some embodiments, transmitting the volume value and time-of-day value to the computing device includes transmitting the associated pairings of {time-of-day value, volume value}. In some embodiments, transmitting the volume value and time-of-day value to the computing device includes transmitting an associated trio of {device-operating-condition value, time-of-day value, volume value}. In some embodiments, transmitting the volume value and time-of-day value to the computing device includes wirelessly transmitting from the console 130 to the computing device. Wireless communication modalities can include Wi-Fi, Bluetooth, Near Field Communications (NFC), cellular Global System for Mobile Communication (“GSM”), electromagnetic (EM), radio frequency (RF), combinations thereof, or the like. In some embodiments, transmitting the volume value and time-of-day value to the computing device includes transmitting the volume value and time-of-day value as each is measured or determined. In some embodiments, transmitting the volume value and time-of-day value to a computing device includes transmitting the associated volume value and time-of-day value pairing at the end of a user-defined time interval. In some embodiments, transmitting the associated volume value and time-of-day value pairing at includes transmitting the associated trio of {device-operating-condition value, time-of-day value, volume value} at the end of the user-defined time interval. In some embodiments, transmitting the volume value and the time-of-day value to the computing device includes transmitting the volume value and time-of-day value before the volume of voided urine is disposed out of the container 110.

While some particular embodiments have been disclosed herein, and while the particular embodiments have been disclosed in some detail, it is not the intention for the particular embodiments to limit the scope of the concepts provided herein. Additional adaptations and/or modifications might appear to those of ordinary skill in the art, and, in broader aspects. these adaptations and/or modifications are encompassed as well. Accordingly. departures can be made from the particular embodiments disclosed herein without departing from the scope of the concepts provided herein.

What is claimed is:

1. An automated urinary output (“UO”)-measuring system, comprising:
a fluid container including a console coupled to one or more ultrasonic sensors and one or more accelerometers, the console including a processor configured to:
receive accelerometer values from the one or more accelerometers;
determine an acceleration state of the fluid container;
activate the one or more ultrasonic sensors when the acceleration state of the fluid container is below a threshold; and
measure a volume of fluid in the fluid container when the acceleration state of the fluid container is zero;
a removable valve coupled to the fluid container; and
an attachment device including a pocket configured to accommodate the fluid container.

2. The automated UO-measuring system according to claim 1, wherein the console further comprises a non-transitory storage medium, an energy source, and one or more logic modules.

3. The automated UO-measuring system according to claim 1, further comprising tubing coupled to the valve, the tubing configured to transmit urine expelled from a user to the fluid container.

4. The automated UO-measuring system according to claim 1, wherein the console, the one or more ultrasonic sensors, the one or more accelerometers, and the valve are organized into a panel.

5. The automated UO-measuring system according to claim 4, wherein the panel divides the fluid container into a proximal section and a distal section.

6. The automated UO-measuring system according to claim 4, wherein the fluid container includes a pump configured to create a low-pressure environment inside the fluid container.

7. The automated UO-measuring system according to claim 6, wherein the processor is configured to:
receive ultrasonic sensor values from the one or more ultrasonic sensors;
correlate the ultrasonic sensor values with a volume-of-voided-urine value in the fluid container and a time-of-day value for a correlation;
determine the volume of voided urine using the ultrasonic sensor values; and
activate the pump to create and maintain the low-pressure environment inside the container.

8. The automated UO-measuring system according to claim 1, wherein the attachment device is configured to be detachably secured to a user.

9. The automated UO-measuring system according to claim 8, wherein the attachment device includes a pair of extensions configured to wrap around an appendage of the user.

10. The automated UO-measuring system according to claim 9, wherein the pair of extensions comprise a pair of fastening arms and a corresponding pair of securing arms.

11. The automated UO-measuring system according to claim 9, wherein the pair of extensions is configured to be secured to the appendage of the user by hook-and-loop fasteners or magnets.

12. The automated UO-measuring system according to claim 1, wherein the attachment device comprises a compression sock configured to be slidably secured to an appendage of a user.

13. The automated UO-measuring system according to claim 1, wherein the fluid container is rigid.

14. A method of automatically measuring urine output, comprising:
coupling an attachment device to a user, the attachment device including a pocket;
inserting a fluid container into the pocket, the fluid container including a console coupled to one or more ultrasonic sensors and one or more accelerometers, the console including a processor;
collecting a volume of voided urine from the user in the fluid container through a removable valve coupled to the fluid container;
receiving accelerometer values from the one or more accelerometers;
determining an acceleration state of the fluid container;
activating the one or more ultrasonic sensors when the acceleration state of the fluid container is below a threshold; and
measuring a volume of voided urine in the fluid container when the acceleration state of the fluid container is zero.

15. The method according to claim 14, wherein collecting the volume of voided urine from the user comprises maintaining a low-pressure environment in the container via a pump associated with the fluid container.

16. The method according to claim 15, further comprising:
receiving ultrasonic sensor values from the one or more ultrasonic sensors;
correlating the ultrasonic sensor values with a volume-of-voided-urine value in the fluid container and a time-of-day value for a correlation;
determining the volume of voided urine using the ultrasonic sensor values; and
activating the pump to create and maintain the low-pressure environment inside the container.

17. The method according to claim 16, further comprising transmitting the volume-of-voided-urine value and the time-of-day value wirelessly to a separate computing device.

18. The method according to claim 14, wherein measuring the volume of voided urine in the fluid container includes measuring and recording at evenly spaced time intervals.

19. The method according to claim 18, wherein the evenly spaced time intervals are defined by the user.

20. The method according to claim 14, further comprising:
receiving pressure readings from a pump coupled with the console, the pump comprising a pressure sensor; and
activating the pump to create and maintain a low-pressure environment inside the fluid container based on the pressure readings.
